(19) United States
(12) Patent Application Publication
(10) Pub. No.: US 2020/0054279 A1
(43) Pub. Date: Feb. 20, 2020

(54) BANDAGE FOR MONITORING SWELLING AT A SELECTED SITE ON THE BODY OF A PATIENT AND METHOD OF MONITORING SWELLING AT A SELECTED SITE ON THE BODY OF A PATIENT

(71) Applicant: Webb Medical LLC, Philadelphia, PA (US)

(72) Inventors: Steven B. KRUPNICK, Philadelphia, PA (US); David KRUPNICK, Philadelphia, PA (US)

(21) Appl. No.: 16/498,178

(22) PCT Filed: Mar. 20, 2018
(86) PCT No.: PCT/US2018/023232

§ 371 Date: Sep. 26, 2019

Related U.S. Application Data
(63) Continuation-in-part of application No. 15/806,849, filed on Nov. 8, 2017.
(60) Provisional application No. 62/550,913, filed on Aug. 28, 2017.
(60) Provisional application No. 62/481,295, filed on Apr. 4, 2017.
(60) Provisional application No. 62/481,295, filed on Apr. 4, 2017.

(51) Int. Cl. A61B 5/00; A61M 5/168

(52) U.S. Cl. CPC A61B 5/4878; A61B 5/7275; A61B 5/746; A61B 5/6833; A61B 5/0002; A61M 2005/1586; A61B 2560/0214; A61M 2205/18; A61M 2205/8206; A61M 2205/581; A61M 5/168

(57) ABSTRACT

A bandage and method of use for monitoring a selected site, e.g., an IV site, of a patient is provided. The bandage includes a first section, a second section, a bridge section, and associated electronic circuitry. The first section is secured to the skin of a patient on one side of the selected site. The second section is secured to the skin of the patient on the other side of the selected site. The bridge section is expandable. The electronic circuitry includes a detection component, e.g., a breakable electronic link or a strain gauge, mounted on the bridge section. The electronic circuitry provides an electrical signal, e.g., an alarm signal, in response to the expansion of the bridge section as determined by the detection component.

### CROSS-REFERENCE TO RELATED APPLICATIONS

This PCT application claims priority under 35 U.S.C. § 119(e) of U.S. Provisional Patent Application Ser. No. 62/481,295, filed on Apr. 4, 2017, entitled EARLY WARNING BANDAGE FOR INTRAVENOUS BLOWOUT OR INTRAVENOUS INFILTRATION AND METHOD OF MONITORING AN INTRAVENOUS INFUSION SITE, and also claims priority under 35 U.S.C. § 119(e) of U.S. Provisional Patent Application Ser. No. 62/550,913, filed on Aug. 28, 2017, entitled MONITORING BANDAGE FOR DETERMINING THE AMOUNT OF SWELLING AT A SPECIFIED SITE OF A PATIENT'S BODY AND METHODS OF MONITORING A SPECIFIED SITE OF A PATIENT'S BODY TO DETERMINE THE AMOUNT OF SWELLING THEREAT, and also claims priority under 35 U.S.C. § 120 of U.S. patent application Ser. No. 15/806,849, filed on Nov. 8, 2017, entitled EARLY WARNING BANDAGE FOR INTRAVENOUS BLOWOUT OR INTRAVENOUS INFILTRATION AND METHOD OF MONITORING AN INTRAVENOUS INFUSION SITE, which in turn claims priority under 35 U.S.C. § 119(e) of U.S. Provisional Patent Application Ser. No. 62/481,295, filed on Apr. 4, 2017, all of whose entire disclosures are incorporated by reference herein.

### FIELD OF THE INVENTION

The disclosed invention relates generally to bandages and more particularly to bandages for monitoring a selected site, e.g., an intravenous (IV) site, on the body of a patient and methods of using such bandages to monitor such selected sites.

### BACKGROUND OF THE INVENTION

A “blown vein” is a term used to describe what happens to a vein when it ruptures or gets punctured causing blood to leak outside of the vein itself. A vein may blow due to it being perforated by a needle that is too large or inserted incorrectly, or by a needle that is implanted too deeply into the vein causing both sides of the vein to be perforated and leading to possible blood leaking out of either end and potentially leading to infiltration or extravasation. Infiltration occurs when an intravenous fluid or medications leak into the surrounding tissue. There are many reasons why a vein blows. For example, some patients have inherently weak vein walls. There are also some specific age groups that are more prone to developing fragile veins. For example, geriatric patients commonly have weak vein walls due to age-related degeneration of connective tissues. Pediatric patients may also have weak vein walls which have not had sufficient time to develop.

In most cases a blown vein is not dangerous nevertheless when a vein blows it should be treated immediately since fluid infiltration can result in painful swelling of the surrounding tissue. If the medication being administered by the intravenous needle is caustic or otherwise dangerous, such as some chemotherapy drugs, dangerous extravasation can result in permanent damage to tissue surrounding the site of the blown vein.

The occurrence of a blow vein becomes more problematic if the patient is non-responsive or otherwise unable to communicate to his healthcare provider the fact that his/her vein has become blown. Thus, with those types of patients it is necessary to frequently check the status of their IV site. For example, it is common to check the IV site of an infant every thirty minutes or so to ensure that the IV hasn't blown and resulted in swelling. Needless to say, it is a waste of valuable medical resources to have to spend the time checking the status of an IV site. Accordingly, a need exists for a cost-effective means and method for automatically monitoring an IV site to detect the occurrence of a venous blow.

The subject invention addresses that need by providing bandages and methods of use which are simple in construction, low in cost, easy to use and which monitor the state of a venous needle access site (IV site) to provide an alarm signal in the event of a venous blow. Moreover, bandages constructed in accordance with this invention can also be used to monitor the amount of swelling at a selected site at the body of a patient, not necessarily an IV site, to determine the amount of swelling at that site.

### SUMMARY OF THE INVENTION

In accordance with one aspect of this invention there is provided a bandage for use at a selected site, e.g., an IV site, on the body of a patient to determine swelling thereat. The bandage comprises a first section, a second section, a bridge section, and electronic circuitry. The first section is configured to be secured to the skin of the patient at a first securement location adjacent the selected site. The second section is configured to be secured to the skin of the patient at a second securement location adjacent the selected site. The second location is spaced from the first location. The bridge section interconnects and bridges the first and second sections. The bridge section is expandable and configured to be expanded or stretched if the spacing between the first and second sections increases as a result of swelling at the selected site. The electronic circuitry comprises a detection component mounted on the bridge section. The electronic circuitry is configured to provide an electrical signal in response to the expansion of the bridge section as detected by the detection component.

In accordance with one preferred aspect of the bandage of this invention, the bandage is an alarm bandage and the selected site is an IV site at which an intravenous needle extends into a vein of the patient at the IV site. The detection component comprises a breakable link configured to break or otherwise rupture in the event that the bridge section expands or stretches beyond a predetermined threshold, whereupon the electrical signal is provided.

In accordance with another preferred aspect of the bandage of this invention, the electronic circuitry additionally comprises an electrical power supply and wherein one of the first section and the second section includes the electrical power supply.

In accordance with another preferred aspect the bandage of this invention the electronic circuitry additionally comprises an alarm producing member for producing an alarm in response to the electrical signal.

In accordance with another preferred aspect the bandage of this invention the expandable section comprises an elastic cloth.

In accordance with another preferred aspect the bandage of this invention the electronic circuitry is comprises a printed circuit.

In accordance with another preferred aspect the bandage of this invention the electrical power supply is a printed battery.

In accordance with another preferred aspect the bandage of this invention the alarm producing member is a printed speaker.

In accordance with another preferred aspect the bandage of this invention the electronic circuitry additionally comprises a separate unit including an electrical power supply and an alarm producing member. The separate unit is configured to be releasably electrically connected to the bandage.

In accordance with another preferred aspect the bandage of this invention the bandage is configured to provide a wireless electrical alarm signal from the bandage to a remote location.

In accordance with another preferred aspect the bandage of this invention the separate unit is configured for wirelessly transmitting the alarm signal to a remote location.

In accordance with another preferred aspect the bandage of this invention the first and second sections are configured for adhesive securement to the skin of the patient.

In accordance with another preferred aspect the bandage of this invention the bandage is a monitoring bandage and the detection component comprises a strain gauge configured to determine the amount that the bridge section expands or stretches. The electronic circuitry is configured to provide the electrical signal indicating the amount that the bridge section expands or stretches as determined by the strain gauge.

In accordance with another preferred aspect the bandage of this invention the bridge section is also configured to be contracted if the spacing between the first section and the second section decreases, and wherein the strain gauge is configured to determine the amount that the bridge section contracts. The electronic circuitry is configured to provide the electrical signal indicating the amount that the bridge section contracts as determined by the strain gauge.

In accordance with another preferred aspect the bandage of this invention the first section and the second section form respective portions of a sleeve configured to be placed around the selected site of the patient's body.

Another aspect of this invention is a method of monitoring a selected site on the body of to provide an electrical signal in the event of swelling of tissue at the selected site. The method comprises providing a bandage that comprises a first section, a second section, an expandable bridge section interconnecting the first and second sections, and electronic circuitry. The electronic circuitry comprises a detection component mounted on the bridge section. The electronic circuitry is configured to provide an electrical signal in response to the expansion or stretching of the bridge section as detected by the detection component. The first section of the bandage is secured onto the skin of a patient adjacent the selected site. The second section of the bandage is secured onto the skin of the patient adjacent the selected site, whereupon the bridging section is disposed over the selected site. Electrical power is applied to the electronic circuitry, whereupon in the event of swelling of the tissue at the selected site the first and second sections move apart to stretch or expand the bridge section, which action is detected by the detection component, whereupon the electronic circuitry provides an electrical signal in response thereto.

In accordance with one preferred aspect of the method of this invention the selected site is an IV site and the electrical signal is provided if the stretching or expansion of the bridge section exceeds a predetermined threshold indicative of an IV blow-out or infiltration.

In accordance with another preferred aspect of the method of this invention the detection component comprises a breakable link configured to break or otherwise rupture in the event that the bridge section expands or stretches beyond the predetermined threshold, whereupon the electrical signal is produced.

In accordance with another preferred aspect of the method of this invention the electrical signal is wirelessly transmitted to a remote location as an alarm signal.

In accordance with another preferred aspect of the method of this invention the bridge section is pre-stretched to a point just below the predetermined threshold before the bandage is secured to the skin of the patient.

In accordance with another preferred aspect of the method of this invention the first and second sections are adhesively secured to the skin of the patient.

In accordance with another preferred aspect of the method of this invention the detection component comprises a strain gauge mounted on the bridge section, wherein the strain gauge determines the amount that the bridge section expands or stretches, whereupon the electronic circuitry provides the electrical signal indicating the amount that the bridge section has expanded or stretched as determined by the strain gauge.

In accordance with another preferred aspect of the method of this invention the bridge section is also configured to be contracted if the spacing between the first section and second section decreases, and wherein the strain gauge determines the amount that the bridge section contracts, whereupon the electronic circuitry provides the electrical signal indicating the amount that the bridge section has contracted as determined by the strain gauge.

In accordance with another preferred aspect of the method of this invention the selected site comprises an IV site, wherein the bandage is secured to the patient's body at the IV site to monitor the condition thereof and to provide an alarm signal in the event of swelling at the IV site indicating an IV blow-out or infiltration.

In accordance with another preferred aspect of the method of this invention the selected site is a limb or the head of the patient, and wherein the monitoring bandage is placed on the patient's body at the selected site to monitor the amount of swelling thereat.

### BRIEF DESCRIPTION OF THE DRAWING FIGURES

FIG. 1 is top plan view of one exemplary embodiment of an alarm bandage constructed in accordance with one aspect of this invention, with the alarm bandage being shown mounted on the hand of a patient receiving an intravenous infusion to provide a warning or alarm in the event of an IV blow-out or infiltration at the IV site which causes swelling at that site;

FIG. 2 is a slightly enlarged top plan view of the bandage shown in FIG. 1;

FIG. 3 is a sectional view taken along line 3-3 of FIG. 2;

FIG. 4 is a top plan view, similar to FIG. 1, but showing an alternative exemplary embodiment of an alarm bandage constructed in accordance with this invention and forming a portion of an IV blow-out or infiltration alarm system;

FIG. 5 is an enlarged top plan view of the bandage shown in FIG. 4;

FIG. 6 is top plan view of one exemplary embodiment of a monitoring bandage constructed in accordance with one aspect of this invention, with the monitoring bandage being shown mounted on the hand of a patient at an IV site to provide a warning or alarm in the event of an IV blow-out or infiltration at the IV site which causes swelling at that site;

FIG. 7 is a slightly enlarged top plan view of the bandage shown in FIG. 6;

FIG. 8 is a sectional view taken along line 8-8 of FIG. 7;

FIG. 9 is a top plan view, similar to FIG. 7, but showing an alternative exemplary embodiment of a monitoring bandage constructed in accordance with this invention;

FIG. 10 is an enlarged top plan view of the monitoring bandage shown in FIG. 9;

FIG. 11 is an illustration of the exemplary embodiment of a monitoring bandage of FIG. 6 shown mounted on the lower limb of a patient to monitor the amount of swelling at that limb; and

FIG. 12 is an illustration, similar, to FIG. 11, but showing an alternative embodiment of a monitoring bandage constructed in accordance with this invention shown mounted on the lower limb of a patient to monitor the amount of swelling at that limb.

### DETAILED DESCRIPTION OF A PREFERRED EMBODIMENT

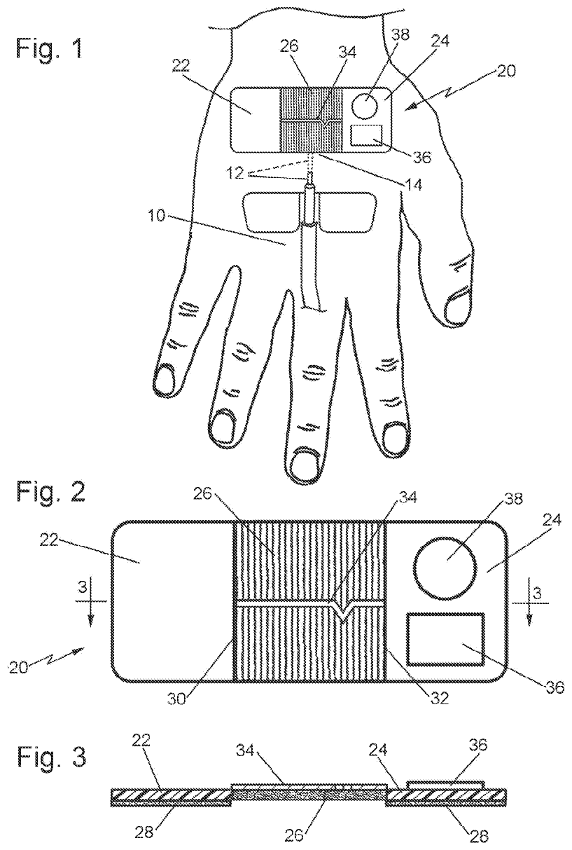

Referring now to the drawings wherein like characters refer to like parts, there is shown in FIG. 1 an exemplary embodiment of an alarm bandage 20 constructed in accordance with this invention shown in one typical application, e.g., at an IV site. The details of the bandage 20 will be described shortly. Suffice for now to state that the bandage 20 is configured to be secured, e.g., adhesively secured, to the skin of a patient at the IV site to automatically monitor the status of swelling at the IV site and provide an alarm signal in the event that the vein in which the needle extends blows, which will result in swelling of adjacent tissue. In the exemplary embodiment shown in FIG. 1, the bandage 20 is located on the back of the hand 10 of a patient at an IV site 14 into which an intravenous (IV) needle 12 extends, and disposed over the immediately adjacent area which will likely become swollen in the event that the vein into which the needle 12 extends blows.

The details of the bandage 20 are best seen in FIGS. 2 and 3. Thus, as can be seen, the bandage 20 basically comprises a first side section 22, a second side section 24 and a bridge section 26. The first and second side sections are each formed of any suitable material, e.g., like that of a conventional adhesive bandage, or a knitted or woven fabric or a non-woven material. In the exemplary embodiment shown the side sections 22 and 24 are each formed of a patch of a woven fabric, plastic (PVC, polyethylene or polyurethane), or latex strip. The material making up the side sections may or may not be waterproof and can be air-tight, if desired. The underside of each side section is in the form of a layer 28 of a conventional skin-engaging adhesive, such as but not limited to, acrylate, including methacrylates and epoxy diacrylates (which are also known as vinyl resins), or any other suitable adhesive materials.

The bridge section 26 is in the form of an expandable or stretchable web of any suitable elastic material, e.g., elastic cloth. The bridge section is fixedly secured to the first side section 22 at a joint 30 and is fixedly secured to the second side section 24 at a joint 32. A detection component, in the form of a breakable link portion 34, of electronic circuitry (to be described shortly) is located on the bridge section 26. In the exemplary embodiment the portion 34 is in the form of a printed circuit, e.g., a link of electrically conductive ink. The link 34 is printed on and extends across the width of the bridging section 26 and is configured to break or rupture in the event that the bridging section expands to a point in excess of a predetermined threshold. The electronic circuitry also includes a power source (e.g., a battery) 36, a loudspeaker 38, and associated circuitry (not shown) interconnecting the link, the battery and the loudspeaker to each other. The electronic circuitry is configured to automatically provide an audible alarm signal from the loudspeaker 38 in the event that the link 34 ruptures or otherwise breaks.

Use of the bandage 20 is as follows. The bandage is applied to the skin of the patient at the location of the IV site 14 by pre-stretching the bridge section 26 to a point just below the point at which the conductive ink link 34 will rupture or otherwise break. With the bridge section pre-stretched, the bridge section is juxtaposed over the IV site and the adhesive underside 28 of the first side section 22 of the bandage is brought into engagement with the skin of the patient on one side of the IV site and the adhesive underside 28 of the second side section 24 of the bandage is brought into engagement with the skin of the patient on the other side of the IV site. Accordingly, the pre-stretched bridge portion 26 of the bandage will be located over the IV site 14, like shown in FIG. 1, and hence will be susceptible to being stretched or otherwise expanded in the event that the tissue at the IV site swells, as would occur if the vein into which the IV needle 12 extends should become blown. In that event, the additional stretching of the bridge section 26 will result in the rupture or breaking of the printed circuit link 34, whereupon the audible alarm signal will be produced by the loudspeaker

It should be pointed out at this juncture that the components making up the electronic circuitry, e.g., the printed link 34, the printed battery 36 and the printed loudspeaker 38 are merely exemplary of various components that can be used, in the bandage providing that some type of alarm signal is automatically produced by the circuitry in the event that the bridge section is stretched or otherwise expanded beyond a predetermined threshold. Moreover, the alarm need not be an audible alarm. Thus, the alarm can be a visual alarm. In fact, the alarm may be both audible and visual. Moreover, the alarm (whatever type it is) need not be produced at the location of the bandage itself, but may be transmitted either wirelessly or otherwise to some remote location, e.g., a nurse's station, to alert personnel thereat that the IV site needs to be checked since it has likely blown.

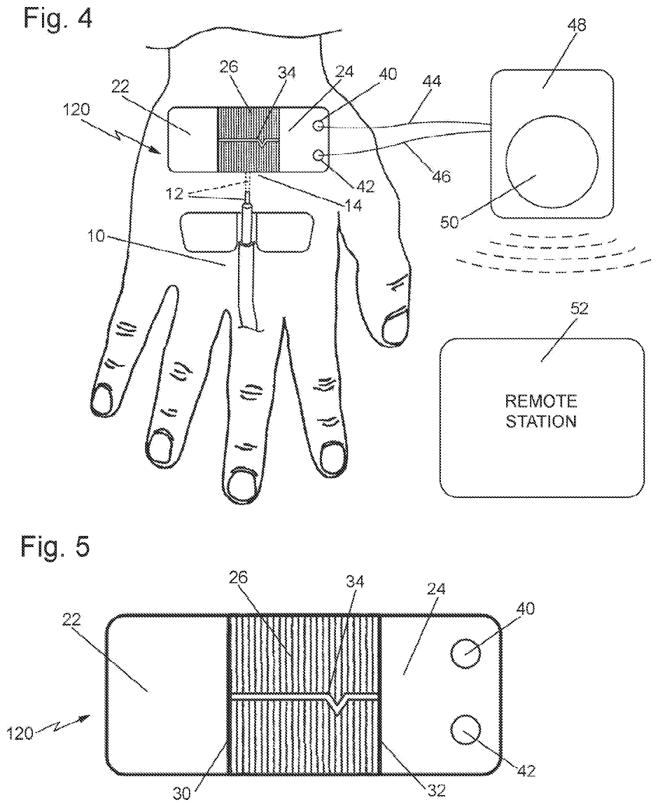

In FIG. 4 there is shown an alternative embodiment of an alarm bandage 120 constructed in accordance with this invention. The bandage 120 is identical in many respects to the bandage 20, except for the details of the electronic circuitry which provides the alarm signal. In the interest of brevity the components of the bandage 120 which are identical to the components of the bandage 20 will be given the same reference numbers and all the details of their construction and operation will not be reiterated. Thus, as can be seen in FIGS. 4 and 5 the bandage 120 basically comprises a first side section 22, a second side section 24 and a bridge section 26. The underside of each side section 22 and 24 is in the form of a layer 28 of a conventional skin-engaging adhesive. The bridge section is fixedly secured to the first side section 22 at a joint 30 and is fixedly secured to the second side section 24 at a joint 32. A breakable link portion 34 is printed on and extends across the width of the bridging section 26 and is configured to break or rupture in the event that the bridging section expands to a point in excess of a predetermined threshold. The side section 24 includes a pair of electrical connectors 40 and 42 which form portions of the electronic circuitry of the bandage. The connectors 40 and 42 are electrically connected to opposite sides of the breakable conductive link 34 by electrical conductors (not shown). The connectors 40 and 42 are configured to be connected to electrical conductors 44 and 46, respectively, of a separate unit 48. The unit 48 includes the remainder of the electronic circuitry of the bandage and basically comprises a housing in which a battery (not shown) for powering the electronic circuitry is located. The unit 48 also serves to house a loudspeaker 50. The loudspeaker is configured to provide an audible alarm in the event that the breakable link 34 ruptures or otherwise breaks, like described above. The unit 48 can be of an alternative construction. Thus, for example, the alarm provided by the unit 48 need not be an audible alarm, but can be a visual alarm or a combination of an audible and visible alarm. Moreover, the unit 48 may he constructed to transmit the alarm wirelessly or by some other means to a remote station 52.

It must be pointed out at this juncture that numerous other variations to the alarm bandages from those specifically disclosed above are contemplated. For example, the printed circuit (breakable) link 34 may be constructed so that it is micro-perforated, thereby making it easier to break when the bridge section is expanded. Moreover, the length of the side sections 22 and 24 of the bandage can be chosen for the particular anatomy to which they will be adhesively secured. For example, one or both of the side sections 22 and 24 can be considerably longer in length than the exemplary embodiments shown in FIGS. 1 and 4, so that one side section would be adhesively secured to the skin of the patient just laterally of the IV site, whereas the other side section would extend around the hand or arm of the patient (depending upon the location of the IV site) to the opposite side. That configuration should ensure that the bandage will not “float” on top of any swelling at the IV site. As should be appreciated by those skilled in the art if the bandage was to “float” on the swelling at the IV site the spacing between the side sections 22 and 24 will remain constant or not increase significantly, whereupon there will be little or no expansion of the bridge section and hence no alarm produced. Instead, with one side section of the bandage adhesively secured on one side of the IV site at a first location and with the other side section adhesively secured on the other side of the IV site at a second location which is more remote from the IV site than the first location, there will be a greater likelihood that in the event of an IV blow and swelling at the IV site, the spacing between the two side sections 22 and 24 will increase sufficiently, thereby resulting in the expansion of the bridge section and the concomitant breakage of the link 34, thereby resulting in the production of the alarm signal.

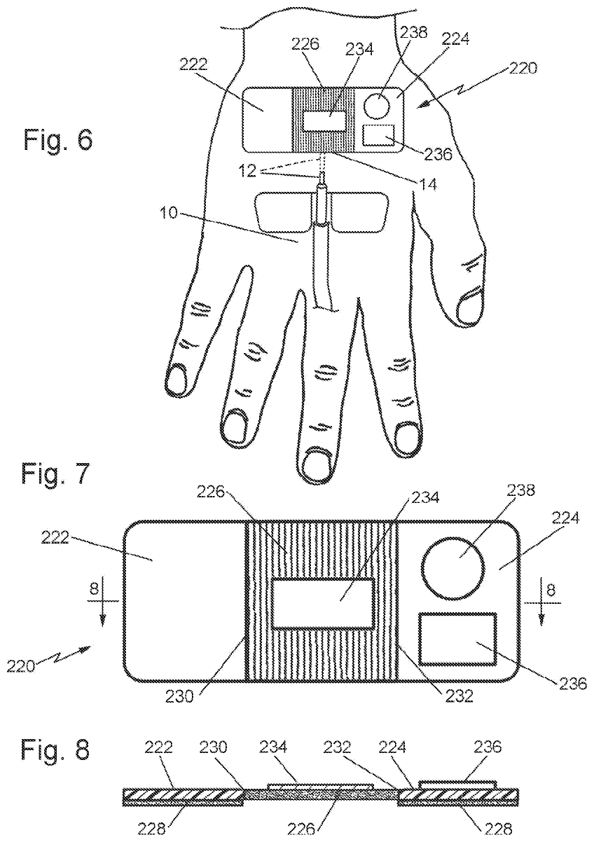

Turning now to FIG. 6 there is shown an exemplary embodiment of a monitoring bandage 220 constructed in accordance with this invention shown in one typical application. The details of the bandage 220 will be described shortly. Suffice for now to state that the bandage 220 is configured to monitor the amount of swelling at a selected site on the body of a patient and to provide a signal indicative thereof. That signal may be used to provide an alarm when the bandage is used to monitor an IV site and that site swells due to an IV blow-out or infiltration. In fact, as will be discussed later, the monitoring bandage 220 can he used to merely monitor the amount of swelling or reduction in swelling at any selected site on the patient's body, not necessarily an IV site. In any case, the monitoring bandage 220 is configured to be secured, e.g., adhesively secured, to the skin of a patient at the selected site to determine the amount of swelling at that site.

In the exemplary embodiment shown in FIG. 6, the selected site is an IV site, and wherein the bandage is configured to automatically monitor the swelling status of the IV site and provide an alarm signal in the event that the vein in which the needle extends blows or the tissue adjacent the monitoring site becomes infiltrated with the IV fluid, which action will result in concomitant swelling at the IV site. Thus, in the exemplary embodiment shown in FIG. 6, the bandage 220 is located on the back of the hand 10 of a patient in the same manner as bandages 20 and 120 as described above.

The details of the bandage 220 are best seen in FIGS. 7 and 8. Thus, as can be seen, the bandage 220 basically comprises a first side section 222, a second side section 224 and a bridge section 226. The first and second side sections are each formed of any suitable material, like that of a conventional adhesive bandage. Thus, in the exemplary embodiment shown the side sections 222 and 224 are each formed of a patch of a woven fabric, plastic (PVC, polyethylene or polyurethane), or latex strip. The material making up the side section may or may not be waterproof and can be air-tight, if desired. The underside of each side section is in the form of a layer 228 of a conventional skin-engaging adhesive, such as but not limited to, acrylate, including methacrylates and epoxy diacrylates.

The bridge section 226 is in the form of an expandable or stretchable web of any suitable elastic material, e.g., elastic cloth. The bridge section is fixedly secured to the first side section 222 at a joint 230 and is fixedly secured to the second side section 224 at a joint 232. A detection component, in the form of a strain gauge 234, is located on the bridge section. In the exemplary embodiment the strain gauge 234 is located on the outer surface of the bridge section, but can be located on the inner surface of the bridge section, if desired. The strain gauge forms a portion of electronic circuitry, and is configured to provide an electrical signal responsive to the force applied on it. The strain gauge can be any suitable type of conventional device, e.g., resistive, capacitive, etc., which is somewhat flexible inasmuch as it will form a portion of a bandage which is arranged to he secured onto some portion of a patient's body. To that end, the strain gauge can be formed of any suitable material capable of affordable mass production, e.g., printing on a flexible substrate. Examples of such materials are: silicon nano-membranes, silver nanoparticle ink, thin films of carbon nanotubes, graphene films, metallic nanoparticles, and other materials that are capable of measuring deformations as large as 100-150% with a relatively large gauge factor, e.g., up to 30 or greater.

The electronic circuitry also includes a power source (e.g., a battery) 236, a loudspeaker 238, and associated circuitry (not shown) interconnecting the strain gauge, the battery and the loudspeaker to each other. The electronic circuitry is configured to automatically provide an audible alarm signal from the loudspeaker 238 in the event that the strain gauge determines that the selected site of the patient's body over which the strain gauge is located has swelled beyond a predetermined threshold value, thereby indicating the likelihood of a blown vein or other infiltration of the IV fluid into adjacent tissue at the IV site. In fact, the strain gauge and its associated electronic circuit of the bandage 220 may be configured to provide an electrical output signal indicative of the amount of swelling at the IV site irrespective of whether or not it exceeds a predetermined threshold, e.g., an alarm value.

Use of the bandage 220 is as follows. The bandage is applied to the skin of the patient at the location of the IV site 14. In particular, the bridge section is juxtaposed over the IV site and the adhesive underside 228 of the first side section 222 of the bandage is brought into engagement with the skin of the patient on one side of the IV site and the adhesive underside 228 of the second side section 224 of the bandage is brought into engagement with the skin of the patient on the other side of the IV site. Accordingly, the bridge portion 226 of the bandage will be located over, i.e., bridge, the IV site 14, like shown in FIG. 6, and hence will be susceptible to being stretched or otherwise expanded in the event that the tissue at the IV site swells, as would occur if the vein into which the IV needle 12 extends should become blown or if the adjacent tissue is infiltrated by the IV fluid. In that event, the stretching of the bridge section 226 will result in the application of a force onto the strain gauge, whereupon the strain gauge will provide an electrical output signal indicative thereof. That signal will be utilized by the electronic circuitry to produce an alarm by the loudspeaker if the amount of force detected is above a predetermined threshold, which threshold is chosen to represent an amount of swelling indicative of an IV blow or tissue infiltration.

It should be pointed out at this juncture that the components making up the electronic circuitry, e.g., the strain gauge 234, the printed battery 236 and the printed loudspeaker 238 are merely exemplary of various components that can be used, in the bandage 220 providing that some type of alarm signal is automatically produced by the circuitry in the event that the bridge section force read by the strain gauge exceeds a predetermined threshold. Moreover, the alarm need not be an audible alarm. Thus, the alarm can be a visual alarm. In fact, the alarm may be both audible and visual. Moreover, the alarm (whatever type it is) need not be produced at the location of the bandage itself, but may be transmitted either wirelessly or otherwise, e.g., a LAN, to some remote location, e.g., a nurse's station, to alert personnel thereat that the IV site needs to be checked since it has likely blown.

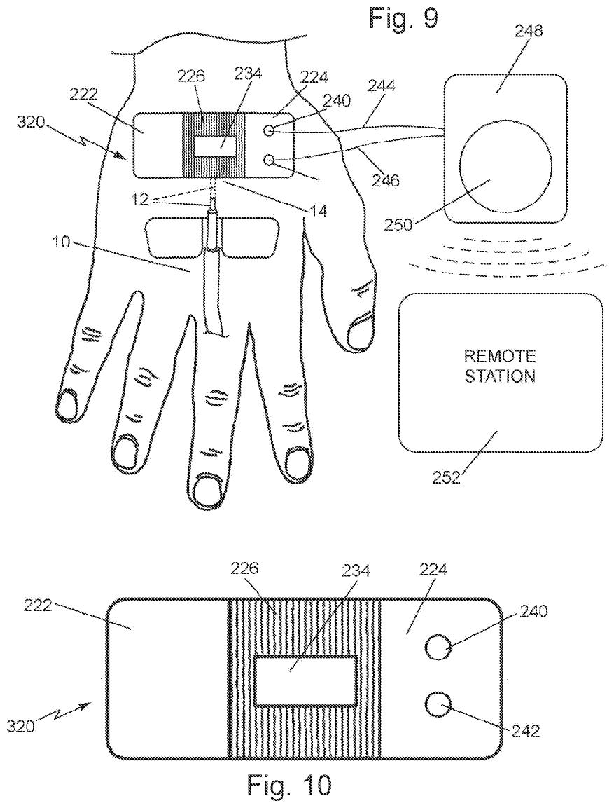

In FIG. 9 there is shown still another alternative embodiment of a monitoring bandage 320 constructed in accordance with this invention. The bandage 320 is identical in many respects to the monitoring bandage 220, except for the details of the electronic circuitry which provides the alarm signal. In the interest of brevity the components of the bandage 320 which are identical to the components of the bandage 220 will be given the same reference numbers and all the details of their construction and operation will not be reiterated. Thus, as can be seen in FIGS. 9 and 10 the bandage 320 basically comprises a first side section 222, a second side section 224 and a bridge section 226. The underside of each side section 222 and 224 is in the form of a layer 228 of a conventional skin-engaging adhesive. The bridge section is fixedly secured to the first side section 222 at a joint 230 and is fixedly secured to the second side section 224 at a joint 232. A strain gauge 234 is printed on the bridging section 226 and is configured to provide a signal indicative of the force applied to it. The side section 224 includes a pair of electrical connectors 240 and 242 which form portions of the electronic circuitry of the bandage. The connectors 240 and 242 are electrically connected to the strain gauge 234 by electrical conductors (not shown). The connectors 240 and 242 are configured to be connected to electrical conductors 244 and 246, respectively, of a separate unit 248. The unit 248 includes the remainder of the electronic circuitry of the bandage and basically comprises a housing in which a battery (not shown) for powering the electronic circuitry is located. The unit 248 also serves to house a loudspeaker 250. The loudspeaker is configured to provide an audible alarm in the event that strain gauge detects a force applied to it which is in excess of a predetermined threshold, like described above.

The unit 248 can be of an alternative construction. Thus, for example, the alarm provided by the unit 248 need not be an audible alarm, but can be a visual alarm or a combination of an audible and visible alarm. Moreover, the unit 248 may be constructed to transmit the alarm to a remote station 252.

Further still and quite significantly, since the monitoring bandage 320, like the monitoring bandage 220, is capable of providing a signal indicative of the amount of swelling at the selected site as detected by the strain gauge, the bandages 320 and 220 (and any other bandage constructed in accordance with this invention), need not be used to only provide signals in the event of an alarm condition, e.g., a blown vein. Thus, the monitoring bandages of this invention can be used in any application to provide a signal representing the amount of swelling detected, irrespective of whether or not it exceeds a predetermined threshold or alarm level. In fact, the monitoring bandages of this invention can be used to provide output signals indicative of the lessening of swelling at a selected site of the patient's body.

It must be pointed out at this juncture that numerous other variations to the monitoring bandages from those specifically disclosed above are contemplated. For example, the strain gauge need not be a printed circuit, so long as it is somewhat flexible and mass producible on a relatively low cost basis. Moreover, the length of the side sections 222 and 224 of the bandages 220 and 320 can be chosen for the particular anatomy to which they will be adhesively secured. For example, one or both of the side sections 222 and 224 can be considerably longer in length than the exemplary embodiments shown in FIGS. 6 and 9, so that one side section would be adhesively secured to the skin of the patient just laterally of the IV site, whereas the other side section would extend around the hand or arm of the patient (depending upon the location of the IV site) to the opposite side. That configuration should ensure that the bandage will not “float” on top of any swelling at the IV site. If the bandage was to “float” on the swelling at the IV site the spacing between the side sections 222 and 224 will remain constant or not increase significantly, whereupon there will be little or no expansion of the bridge section and hence no alarm produced. Instead, with one side section of the bandage adhesively secured on one side of the IV site at a first location and with the other side section adhesively secured on the other side of the IV site at a second location which is more remote from the IV site than the first location, there will be a greater likelihood that in the event of an IV blow and swelling at the IV site, the spacing between the two side sections 222 and 224 will increase sufficiently, thereby resulting in the expansion of the bridge section and the concomitant production of the alarm signal.

It should also be pointed out at this juncture, that the electronic circuitry can be set with a predetermined amount of “strain” already on the bandage at the time of its application to the selected site. That action has the effect of zeroing the bandage for the purposes of monitoring swelling at the selected site, which would limit the problems of a patient flexing or moving around during the application process. Making use of a strain gauge to determine the amount of swelling at the selected site allows the doctor or other health care provider to have an acceptable amount of swelling/expansion at the site if there is a special medication or other acceptable reason for a site to experience expansion/swelling during a procedure or IV insertion.

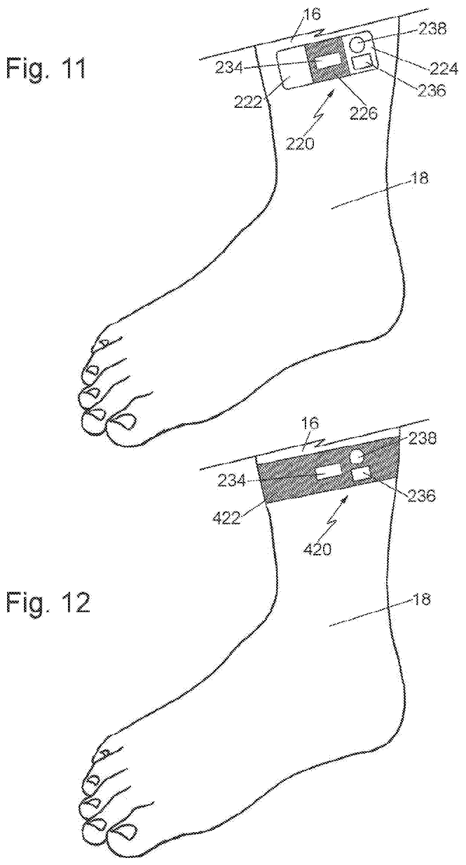

As should be appreciated by those skilled in the art, by utilizing a low cost strain gauge, a monitoring bandage constructed in accordance with this invention can be used for purposes other than detecting vein blowouts and IV infiltrations. Thus, the monitoring bandages of this invention can serve as a diagnostic tool or home care aid for swelling detection. In this regard, the monitoring bandage could be used to monitor a heart or kidney failure patient for swelling of their lower extremities, an early sign of poorly controlled disease, and provide feedback to a medical professional or home care provider. One such application is shown in FIG. 11 wherein the exemplary embodiment of the monitoring bandage 220 of FIG. 6 is shown mounted on the lower limb, e.g., on the calf 16 just above the ankle 18, of a patient to monitor the amount of swelling at that limb. FIG. 12 is an illustration of another exemplary monitoring bandage 420 constructed in accordance with this invention, shown mounted on the lower limb, e.g., on the calf 16 just above the ankle 18 of a patient.

The monitoring bandage 420 is constructed similarly to the monitoring bandage 320 except that instead of making use of first side section 222, a second side section 224 and a bridge section 226, the bandage 420 makes use of a stretchable sleeve or band 422 of any suitable material, e.g., an elasticized fabric or cloth, neoprene, rubber, etc., for encircling the site to be monitored for swelling. Thus the band 422 serves as the first side section, the bridge section, and the second side section. The strain gauge 234 is mounted on that band 422, either on the inside or outside thereof, so that it operates in a manner similar to the strain gauge 234 mounted on the bridge section 226 of the bandage 220. The remainder of the electronic circuit, e.g., the power source (e.g., a battery) 236, the loudspeaker 238, and associated circuitry (not shown) interconnecting the strain gauge, the battery and the loudspeaker to each other are also mounted on the sleeve or band.

In a hospital setting, a monitoring bandage constructed in accordance with this invention can be used to monitor improvement or worsening of a patient's cardiac or renal condition as it relates to their lower extremity edema, thereby helping to determine if the patient is ready for discharge. A key feature of this use being that the monitoring bandage will be monitoring if swelling is decreasing as well as increasing. That ability should provide an important indicator in a physician's decision-making process regarding the health of the patient. Moreover, since the monitoring bandages of this invention are capable of monitoring decreases in swelling, in addition to monitoring increases in swelling, they may act as an important diagnostic device per se for both infants and other patients (geriatric or adult) where swelling has already occurred and the physician or health care provider wants to measure improvement during the patient's treatment.

While the alarm bandages and the monitoring bandages as described above have been in the form of either a strip or a sleeve or band, it should be clear that they could made be in different shapes and sizes to cover various areas for swelling monitoring in addition to the hand, wrist, and calf. Thus, the bandage can be in the form of a large diameter sleeve suitable for encircling the abdominal region of a patient or for encircling the head of a patient.

Moreover, it is contemplated that one could combine an alarm bandage constructed in accordance (e.g., a bandage with a breakable link) with this invention and a monitoring bandage constructed in accordance with this invention (e.g., a bandage with a strain gauge) to form a composite or merged bandage. Such a bandage should act as fail safe solution for monitoring an IV site during any IV procedure whether in a hospital, some other health care facility, a nursing home, etc.

Without further elaboration the foregoing will so fully illustrate our invention that others may, by applying current or future knowledge, adopt the same for use under various conditions of service.

1. A bandage for use at a selected site on the body of a patient to determine swelling thereat, said bandage comprising:
a first section configured to be secured to the skin of a patient at a first securement location adjacent the selected site;
a second section configured to be secured to the skin of a patient at a second securement location adjacent the selected site, said second location being spaced from said first location;
a bridge section interconnecting and bridging said first and second sections, said bridge section being expandable and configured to be expanded or stretched if the spacing between said first and second sections increases as a result of swelling at the selected site; and
electronic circuitry comprising a detection component mounted on said bridge section, said electronic circuitry being configured to provide an electrical signal in response to the expansion of said bridge section as detected by said detection component.

2. The bandage of claim 1 wherein the selected site is an IV site at which an intravenous needle extends into a vein of the patient at the IV site, and wherein said detection component comprises a breakable link configured to break or otherwise rupture in the event that said bridge section expands or stretches beyond a predetermined threshold, whereupon said electrical signal is provided.

3. The bandage of claim 2 wherein said electronic circuitry additionally comprises an electrical power supply and wherein one of said first section and said second section includes said electrical power supply.

4. The bandage of claim 2 wherein said electronic circuitry additionally comprises an alarm producing member for producing an alarm in response to said electrical signal.

5. The bandage of claim 2 wherein said expandable section comprises an elastic cloth.

6. The bandage of claim 2 wherein said electronic circuitry is comprises a printed circuit.

7. The bandage of claim 3 wherein said electrical power supply is a printed battery.

8. The bandage of claim 4 wherein said alarm producing member is a printed speaker.

9. The bandage of claim 2 wherein said electronic circuitry additionally comprises a separate unit including an electrical power supply and an alarm producing member, said separate unit being configured to be releasably electrically connected to said bandage.

10. The bandage of claim 2 wherein said bandage is configured to provide a wireless electrical alarm signal from said bandage to a remote location.

11. The bandage of claim 1 wherein said first and second sections are configured for adhesive securement to the skin of the patient.

12. The bandage of claim 1 wherein said detection component comprises a strain gauge configured to determine the amount that said bridge section expands or stretches, said electronic circuitry being configured to provide said electrical signal indicating the amount that said bridge section expands or stretches as determined by said strain gauge.

13. The bandage of claim 12 wherein said bridge section is also configured to be contracted if the spacing between said first section and said second section decreases, and wherein said strain gauge is configured to determine the amount that said bridge section contracts, said electronic circuitry being configured to provide said electrical signal indicating the amount that said bridge section contracts as determined by said strain gauge.

14. The bandage of claim 1, wherein said first section and said second section form respective portions of a sleeve configured to be placed around the selected site of the patient's body.

15. A method of monitoring a selected site on the body of a patient to provide an electrical signal in the event of swelling of tissue at said selected site, said method comprising:
providing a bandage comprising a first section, a second section, an expandable bridge section interconnecting said first and second sections, and electronic circuitry, said electronic circuitry comprising a detection component mounted on said bridge section, said electronic circuitry being configured to provide said electrical signal in the response to the expansion or stretching of said bridge section as detected by said detection component;
securing said first section of said bandage onto the skin of the patient adjacent said selected site;
securing said second section of said bandage on the skin of the patient adjacent said selected site, whereupon said bridging section is disposed over said selected site; and
applying electrical power to said electronic circuitry, whereupon in the event of swelling of the tissue at said selected site said first and second sections move apart to stretch or expand said bridge section, which action is detected by said detection component, whereupon said electronic circuitry provides an electrical signal in response thereto.

16. The method of claim 15 wherein said selected site is an IV site and said electrical signal is provided if the stretching or expansion of said bridge section exceeds a predetermined threshold indicative of an IV blow-out.

17. The method of claim 16 wherein said detection component comprises a breakable link configured to break or otherwise rupture in the event that said bridge section expands or stretches beyond said predetermined threshold, whereupon said electrical signal is produced.

18. The method of claim 16 wherein said electrical signal is wirelessly transmitted to a remote location as an alarm signal.

19. The method of claim 16 wherein said bridge section is pre-stretched to a point just below said predetermined threshold before said bandage is secured to the skin of the patient.

20. The method of claim 16 wherein said first and second sections are adhesively secured to the skin of the patient.

21. The method of claim 15 wherein said detection component comprises a strain gauge mounted on said bridge section, wherein said strain gauge determines the amount that said bridge section expands or stretches, whereupon said electronic circuitry provides said electrical signal indicating the amount that said bridge section has expanded or stretched as determined by said strain gauge.

22. The method of claim 21, wherein said bridge section is also configured to be contracted if the spacing between said first section and second section decreases, and wherein said strain gauge determines the amount that said bridge section contracts, whereupon said electronic circuitry provides said electrical signal indicating the amount that said bridge section has contracted as determined by said strain gauge.

23. The method of claim 21, wherein said selected site comprises an IV site, wherein said monitoring bandage is secured to the patient's body at said IV site to monitor the condition thereof and to provide an alarm signal in the event of swelling at the IV site indicating an IV blow-out.

24. The method of claim 15, wherein said selected site is a limb or the head of the patient, and wherein said monitoring bandage is placed on said patient's body at said selected site to monitor the amount of swelling thereat.

25. The method of claim 18, wherein said selected site is a limb or the head of the patient, and wherein said monitoring bandage is placed on said patient's body at said selected site to monitor the amount of swelling thereat.

26. The method of claim 21, wherein said selected site is a limb or the head of the patient, and wherein said monitoring bandage is placed on said patient's body at said selected site to monitor the amount of swelling thereat.
